(19) United States
(12) Patent Application Publication
(10) Pub. No.: US 2007/0154390 A1
(43) Pub. Date: Jul. 5, 2007

(54) Nuclear molecular imaging contrast agent

(75) Inventors: Huang Chien Liang, Hsinchu City (TW); Hsin-Ell Wang, Taoyuan County (TW); Chin-I Lin, Tainan County (TW); Shian-Jy Wang, Hsinchu County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/449,764

(22) Filed: Jun. 9, 2006

(30) Foreign Application Priority Data
Dec. 30, 2005 (TW) 94147660

(51) Int. Cl. A61K 51/00; A61K 49/10; C08G 63/91; C08G 63/48

(52) U.S. Cl. 424/1.11; 424/9.34; 525/54.2

(57) ABSTRACT

A nuclear molecular imaging contrast agent. The nuclear imaging contrast agent comprises a polymer according to the structure of

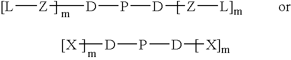

wherein, P is

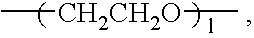

and 1 is not less than 1; D is a C3-30 dendritic moiety having a plurality of oxygen residue; Z is a C3-20 moiety having a plurality of functional groups, wherein the functional groups comprise carbonyl, carboxyl, amine, ester, amide, or chelate group; L is a radioisotope or analyte-specific moiety ; m is not less than two ; and at least one X is an analyte-specific moiety; the other X can be hydrogen atom.

### BACKGROUND OF THE INVENTION

1. Field of the Invention

The present invention relates to a nuclear molecular imaging contrast agent, and more particularly relates to a nuclear molecular imaging contrast agent containing a polymer with dendritic moiety.

2. Description of the Related Art

Currently, medical imaging is capable of creating functional and anatomical image via physical signals of magnetic, photo (fluorescence, near-infrared, X-ray), and radioactive rays emitted by different imaging instruments. The imaging instruments include the Planar X-ray Imaging system, the X-ray Computerised Tomography (CT) system, and the Nuclear Magnetic Resonance Imaging (NMRI) system, which are utilized in the diagnosis of the central nervous system, skeletal nervous system, stomach, ribcage, angiography, diagnosis of biliary tractphotography, and the diagnosis of mutation of tumor tissue. The appearance of anatomic tissue does not change, but the change in blood circulation, cell activity, and metabolism of the location has been occurred in many clinical symptoms. Therefore, the location of the illness can be detected early by high sensitivity Nuclear Imaging.

Currently, radioisotope targeting compounds (conventional radioisotope targeting compounds are compounds with small molecular weight) are injected into a living organism in the majority of nuclear molecular imaging systems, which adopt a detector to monitor and image the biodistribution of the radioisotope targeting compounds. The compounds interact with the molecules in the living organism to yield physiological and biochemical reactions. Thus, detected changes in biochemical functions can be detectable prior to anatomic pathology occurs in the living organism.

The most common application in the clinical nuclear molecular imaging field so far is the diagnosis of malignant tumors. Since most malignant tumors show higher metabolism and usage rate of glucose, amine acid, and deoxyibouncleic acid (DNA), a proper nuclear molecular imaging contrast agent is utilized as a design reference to such characteristics, thus a benign tumor and a malignant tumor can be detected. Furthermore, the nuclear molecular imaging contrast agent can provide a precise and reliable estimation for detection of a curative effect, relapse of a malignant tumor, and metastasis of symptoms. However, conventional nuclear molecular imaging contrast agents belong to the categories of chemical compounds with small molecular weight, which have a strong ability to break through human vascular endothelial cells (HEVC). Thus, the nuclear molecular imaging contrast agents easily flow away and are broadly distributed in the circulation process and can not reach the target location. In addition, these contrast agents with small molecular weight after entering. The living organisms are easily metabolized by human body and can not reach the developing object. Even if the developing agents with small molecular weight reach the developing location, the developing agents can reach the effect of the developing only certain numbers of accumulated developing agents. Therefore, to obtain the efficiency of ideal image contrast agents in portion, the dosage of the nuclear molecular imaging contrast agents must be magnified. Nevertheless, not only does the risk of toxicity caused by highly concentrated radioisotope occur, but an abundance of molecular imaging agents also abundantly accumulate in the same portion. Thus, clinical applications are limited. Accordingly, a desired nuclear molecular imaging contrast agent is developed in order to efficiently target the location of the illness with lower dosage, which is a significant topic of research in Nuclear Imaging technology.

### BRIEF SUMMARY OF THE INVENTION

The invention provides a nuclear molecular imaging contrast agent capable of recognizing an affected part of a human body with high sensitivity. A detailed description is given in the following embodiments with reference to the accompanying drawings.

An embodiment of a nuclear molecular imaging contrast agent is provided. The nuclear imaging contrast agent comprises a polymer according to the structure of formula (I) or formula (II) as below:

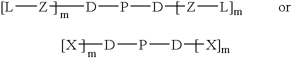

wherein P is

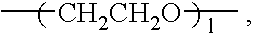

and 1 is not less than 1; D is independent and comprises a C3-30 dendritic moiety with n oxygen residual groups, wherein n is not less than 3, and D respectively binds with P and Z by the oxygen residual groups;

Z is independent and comprises a C3-20 moiety having a plurality of functional groups, wherein the functional groups are selected from a group consisting of carbonyl, carboxyl, amine, ester, amide, or chelate group, and Z respectively bind with D and L by the individual functional group; L is independent and comprises a radioisotope or analyte-specific moiety; m is not less than two and m equals n−1; and at least one X is an analyte-specific moiety, the other X comprise hydrogen atoms. X can be all analyte-specific moieties.

For the polymers contained in the nuclear molecular imaging contrast agent in the embodiments, P can be any conventional binding segment of ethylene glycol and its derivatives, preferably polymer segments of poly ethylene glycol. In some embodiments, D is a C3-30 dendritic moiety with n oxygen residual groups which can be bonded with D and a plurality of Z. Preferably, D is 2,2-dihydroxymethyl propanoic acid and residual groups of the derivative thereof, such as

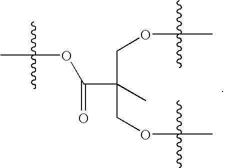

### Besides, D can be a dendrimer moiety with layers of unrestricted numbers, preferably 2 to 3 layers, such as

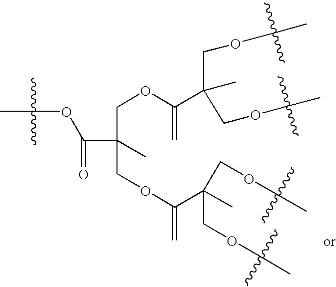

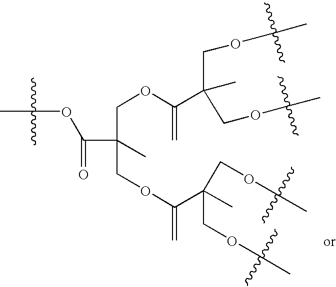

The radioisotope in some embodiments is capable of taking part in physiological metabolism as a developing agent with high sensitivity and precision of nuclear medical fields, such as F-18, In-11, Ga-68, Xe-133, Tc-99m, or Gd-153. According to some embodiments, the analyte-specific moiety is a molecular moiety specifically reacted with a specific target, such as a folic acid group, a glucose group, or an amino acid group. In some embodiments, Z can be a chelated agent, such as a residual group of ethylenedinitrilo tetraacetic acid (EDTA) or a residual group of ethylenediimino dibyric acid (EDBA). In addition, Z can be

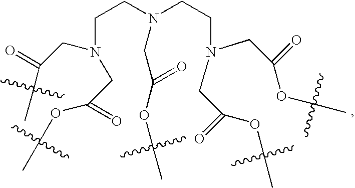

wherein Z bonds with D by one oxygen atom and bonds with L by the other oxygen atoms.

### DETAILED DESCRIPTION OF THE INVENTION

The following description is of the best-contemplated mode of carrying out the invention. This description is made for the purpose of illustrating the general principles of the invention and should not be taken in a limiting sense. The invention is an improved bonding pad and method for their fabrication. Although the invention is described with respect to a specific embodiment, the principles of the invention, as defined by the claims appended herein, can obviously be applied beyond the specifically described embodiments of the invention described herein. Moreover, in the description of the invention, certain details have been left out in order to not obscure the inventive aspects of the invention. The details left out are within the knowledge of a person of ordinary skill in the art.

### EXAMPLE 1

Preparation of polymer (A)

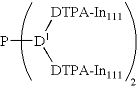

containing radioisotope In₁₁₁:

P: polyethylene glycol segment (molecular weight˜4000);

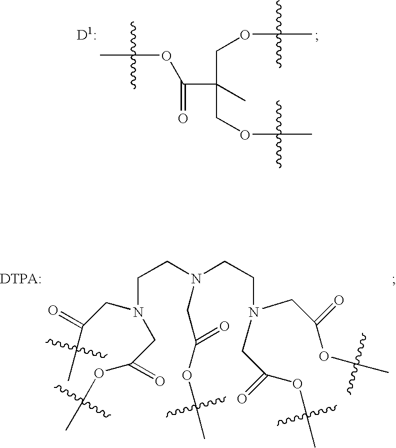

wherein D¹ bonds with a carbonyl group (CO) of DTPA by oxygen residual group to form an ester group (COO).

Preparation procedure of the polymer (A) containing the radioisotope In₁₁₁ is shown as below:

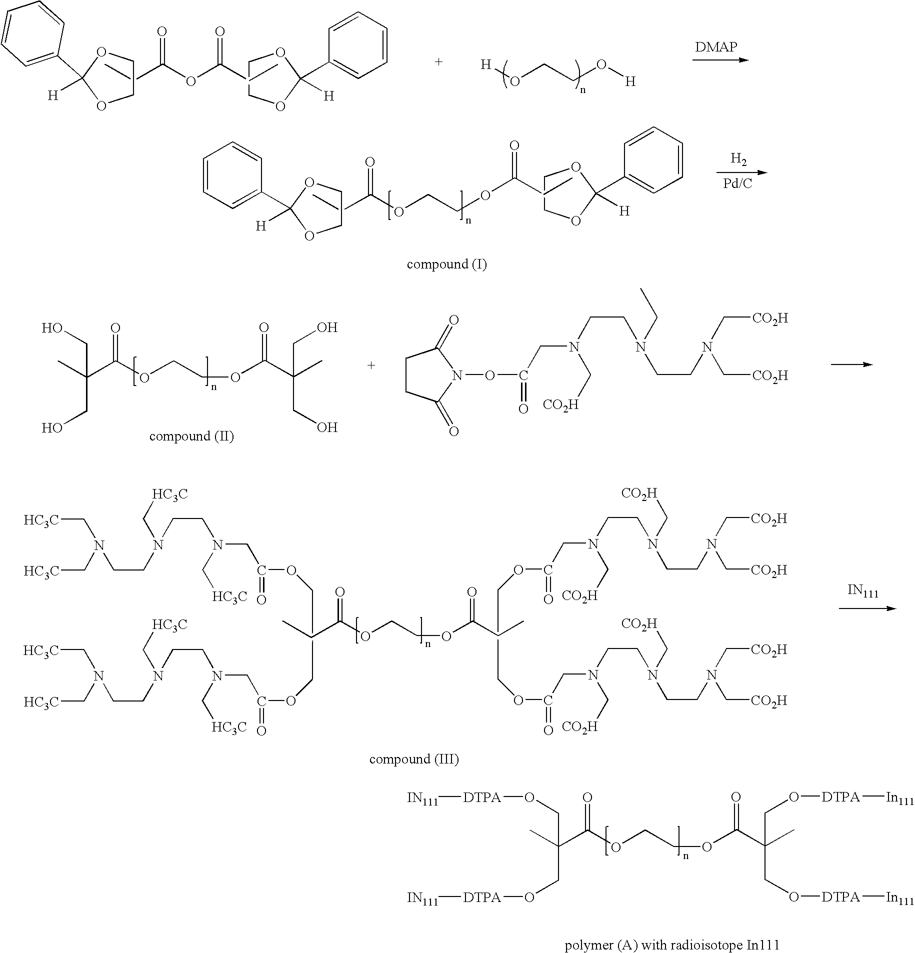

At first, PEG diol (MW 4000 Da, 9.2 g, 2.3 mmol, 1 eq) is mixed with dimethylaminopyridine (DMAP, 0.1670 g, 0.39 mmol) in a round-bottomed conical flask followed by dissolving 25 ml dichloromethane (DCM) in nitrogen atmosphere and filling it in the flask. Thereafter, 4.27g (10 mmol) Benzylidene-2,2-bis(oxymethyl) propionic anhydride dissolved in another flask is gradually added in the reacting round-bottomed conical flask recited above. After stirring for 24 hours in the room temperature, 10 ml methanol is added and the reaction is processed for 6 hours to remove un-reacted Benzylidene-2,2-bis(oxymethyl) propionic anhydrides. Subsequently, overdose (700 mL) ether is also added and continuously stirred to form compound (I), which is white precipitant and yields around 90%. Physical measurement of the compound (I) is listed below:

IR (cm-I): 2890, 1738, 1150.

1H NMR (400 MHz, CDCl3): δ 1.06 (s, 6), 3.55 (t, 6), 3.61 (bs), 3.68 (t, 6), 4.32 (t, 4), 4.64 (d, 4), 5.43 (s, 2), 7.28 (m, 6), 7.42 (m, 4).

Thereafter, 11.8 g compound (I) is dissolved in 40 mL CH₂Cl₂/MeOH(1:2) followed by adding 1.18 g Pd/C and stirring for 24 hours in the saturated hydrogen atmosphere. After the reaction is complete, Pd/C is filtered from DCM. A white precipitant product is rapidly obtained via washing by overdose (600 mL) ether. Thus, a compound (II) with yield of around 90% is formed by cooling and drying. Physical measurement of the compound (II) is listed as below:

IR (cm-I): 3480, 2890, 1725, 1148.

1H NMR (400 MHz, CDCl3): δ 1.08 (s, 6), 3.67 (bs), 4.31 (t, 4)

Thereafter, 2.0 g (0.4618 mmol) compound (II) and 1.0871g (2.2 mmol) diethylenetriaminepentaacetic acid mono-N-hydroxysuccinimide ester (DTPA-HSIE) are disposed in a round-bottomed conical flask of 50 mL and dried by vacuum for 3 hours. Then, 10 mL anhydrous dimethyl sulfoxide (DMSO) and 224 mL Triethylamine are injected followed by continuously stirring for 48 hours in the saturated hydrogen atmosphere. Free DTPA-HSIE is removed by dialysis, spec. of which is MW1000. 1000 mL deionized water is provided each time as dialysis solvent to keep monitoring DTPA-HSIE until it is absent in the dialysis solvent. Ultimately, compound (III) is formed as a white solid product after cooling and drying and physical measurement thereof is listed below:

IR(cm-1): 3446, 2888, 1714, 1638, 1109.

1H NMR (400 MHz, CDCl3): δ 1.14 (s, 6), 3.1 (t, 16), 3.4 (t, 16), 3.57 (bs), 3.75 (s, 8), 3.8 (s, 32).

Finally, the compound (III) is dissolved by water to form a solution with concentration of 0.46×10⁻¹⁰/L. Thereafter, In-111-acac 66 μCi (dissolved in ethanol) is added in the solution followed by evacuating ethanol with nitrogen. The water bath is processed for 20 mins in the temperature of 70° C. Subsequently, reverse phase C18-plus Sep-Pak is executed for separation and purification, and a polymer (A) containing radioisotope In₁₁₁ is then formed.

### EXAMPLE 2

Preparation of polymer (B)

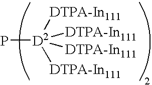

containing radioisotope In₁₁₁:

P: polyethylene glycol segment (molecular weight˜4000);

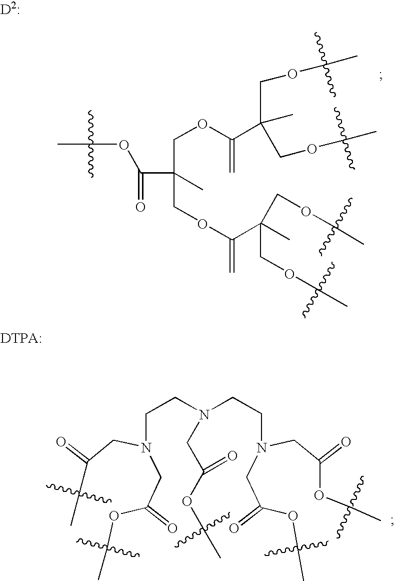

wherein D¹ bonds with a carbonyl group (CO) of DTPA by oxygen residual group to form a ester group (COO).

Preparation procedure of the polymer (B) containing the radioisotope In₁₁₁ is shown as below:

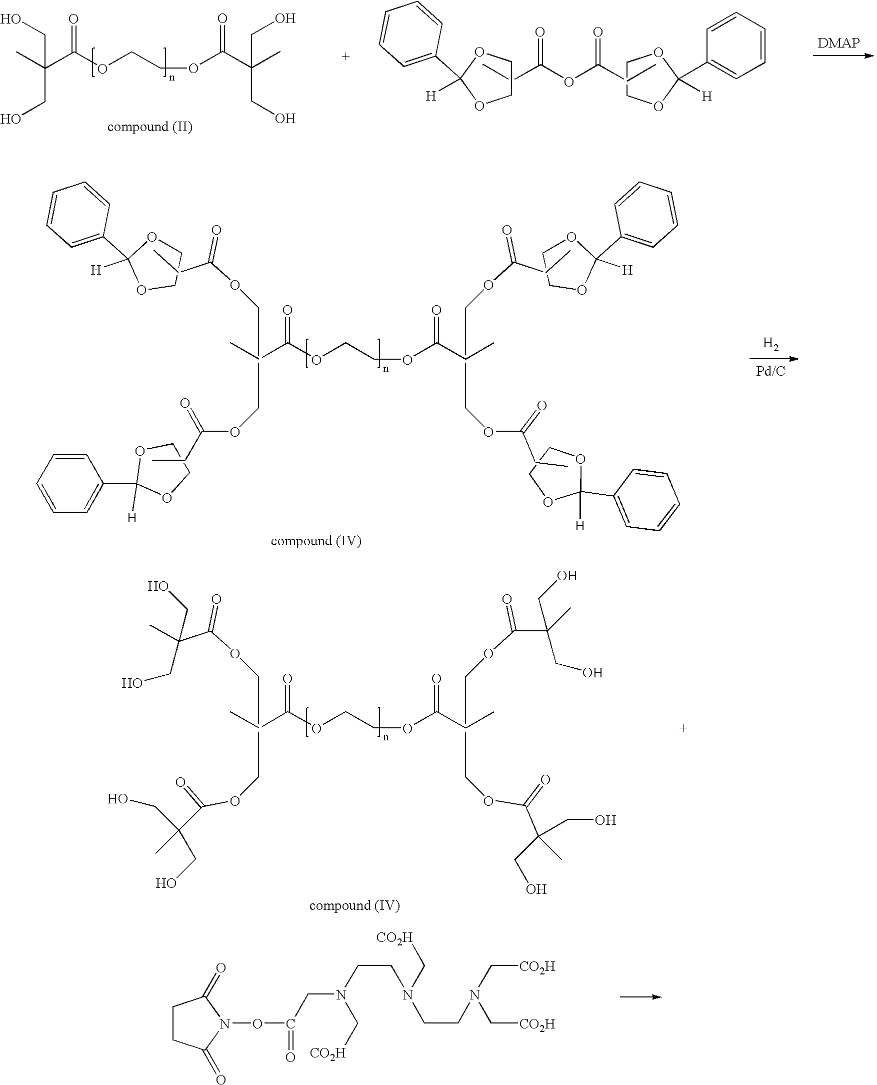

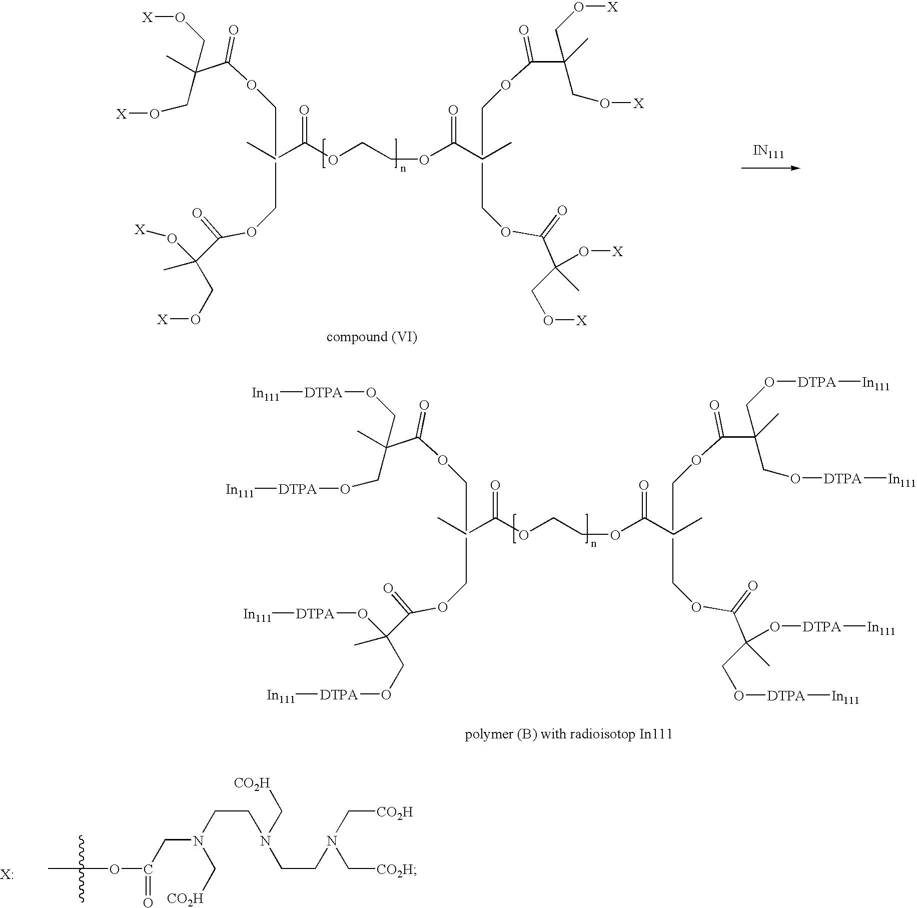

At first, compound (II) (95.6 g, 0.83 mmol) is mixed with dimethylaminopyridine (DMAP, 0.326g, 2.6 mmol) in a round-bottomed conical flask followed by dissolving 25 ml dichloromethane (DCM) in nitrogen atmosphere and filling it in the flask. Thereafter, 5.69 g (10 mmol) Benzylidene-2,2-bis(oxymethyl) propionic anhydride dissolved in another flask is gradually added in the reacting round-bottomed conical flask recited above. After stirring for 24 hours at room temperature, 15 ml methanol is added and the reaction is processed for 6 hours to remove un-reacted Benzylidene-2,2-bis(oxymethyl) propionic anhydrideis. Subsequently, overdose (700 mL) ether is also added and continuously stirred to form compound (IV), which is white precipitant and yields around 80%. Physical measurement of the compound (IV) is listed below:

IR (cm-I): 2885, 1740, 1100.

1H NMR (400 MHz, CDCl3): δ 1.03 (s, 12), 1.26 (s, 6), 3.63 (bs), 3.78 (t, 4), 4.03 (t, 4), 4.38 (s, 8), 4.56 (d, 8), 5.41 (s, 4), 7.19 (m, 12), 7.38 (m, 8).

Thereafter, 5.5 g compound (IV) is dissolved in 45 mL DCM/MeOH(1:2) followed by adding 1.18 g Pd/C and stirring for 24 hours in the saturated hydrogen atmosphere. After the reaction is complete, Pd/C is filtered from DCM. A white precipitant product is rapidly obtained via washing by overdose (600 mL) ether. Thus, a compound (II) with yield of around 90% is formed by cooling and drying. Physical measurement of the compound (V) is listed as below:

IR (cm−1): 3401, 2887, 1727, 1108.

1H NMR (400 MHz, CDCl3): δ 1.03 (s, 12), 1.19 (s,6), 3.43 (m, 8), 3.64 (bs), 4.08 (m, 8), 4.40 (d, 4)

Thereafter, 1.40 g (0.265 mmol) compound (V) and 1.248 g (2.54 mmol) diethylenetriaminepentaacetic acid mono-N-hydroxysuccinimide ester (DTPA-HSIE) are disposed in a round-bottomed conical flask of 50 mL dried by vacuum for 4 hours. Then, 10 mL anhydrous dimethyl sulfoxide (DMSO) and 350 mL Triethylamine are injected followed by continuous stirring for 48 hours in the saturated hydrogen atmosphere. Free DTPA-HSIE is removed by dialysis, spec. of which is MW1000. 1000 mL deionized water is provided each time as dialysis solvent to keep monitoring DTPA-HSIE until it is absent in the dialysis solvent. Ultimately, compound (VI) is formed as a white solid product after cooling and drying and physical measurement thereof is listed below:

IR(cm−1): 3438, 2939, 2678, 1725, 1634, 1228.

1H NMR (400 MHz, CDCl3): δ 1.04 (m), 1.18 (m), 3.07 (t, 16), 3.21 (t, 16), 3.58 (bs), 3.68 (m), 3.79 (d), 4.21 (bs).

Finally, the compound (VI) is dissolved by water to form a solution with concentration of 0.46×10⁻¹⁰/L. Thereafter, In-111-acac 66 μCi (dissolved in ethanol) is added in the solution followed by evacuating ethanol with nitrogen. The water bath is processed for 20 mins in the temperature of 70° C. Subsequently, reverse phase C18-plus Sep-Pak is executed for separation and purification, and then, a polymer (B) containing radioisotope In₁₁₁ is then formed.

### EXAMPLE 3

preparation of polymer (C)

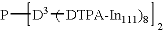

containing radioisotope In₁₁₁:

P: polyethylene glycol segment (molecular weight˜4000);

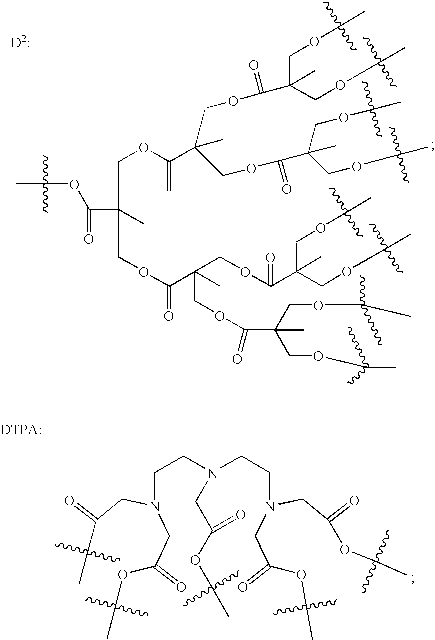

wherein D² bonds with a carbonyl group (CO) of DTPA by oxygen residual group to form a ester group (COO).

Preparation procedure of the polymer (C) containing the radioisotope In₁₁₁ is shown as below:

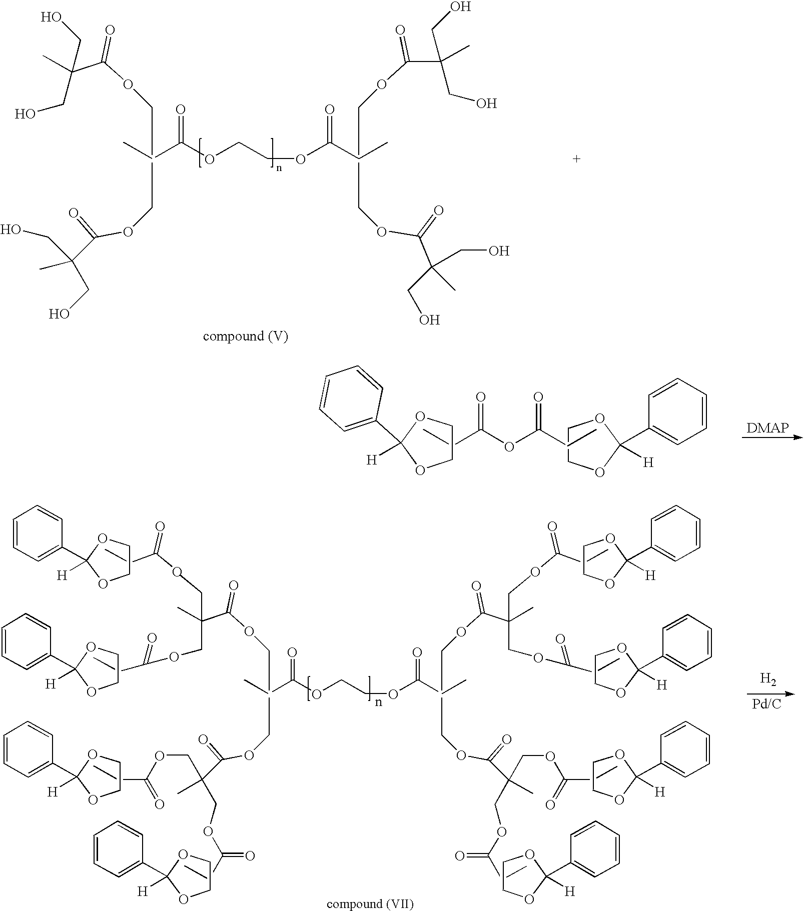

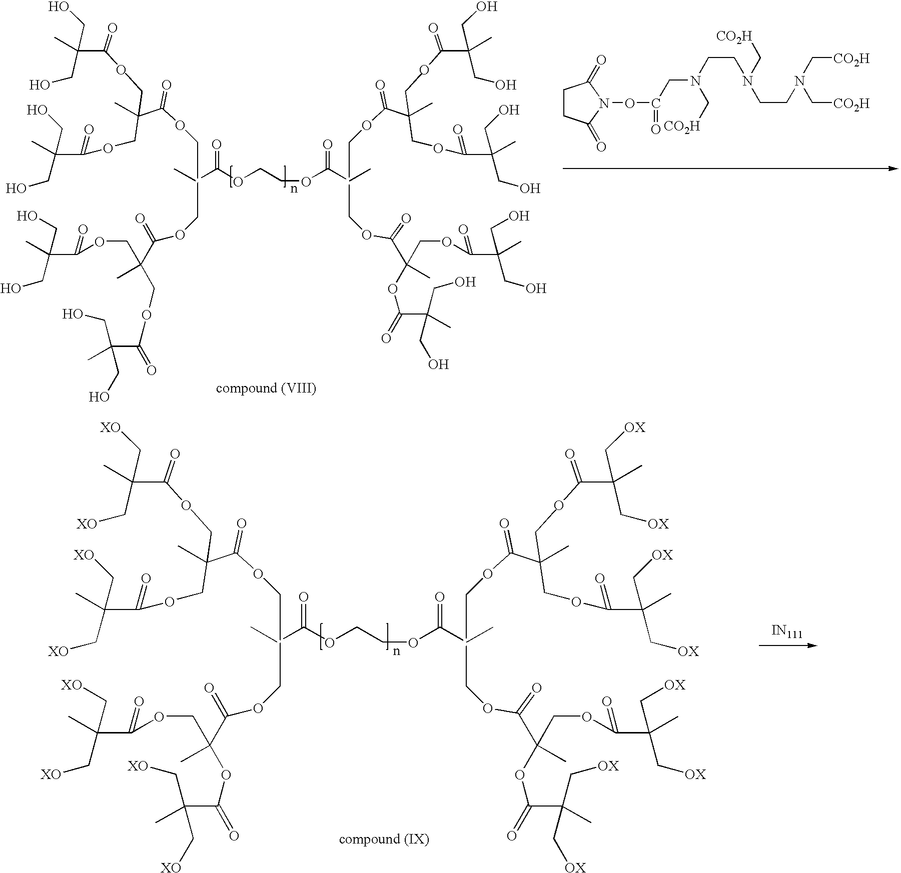

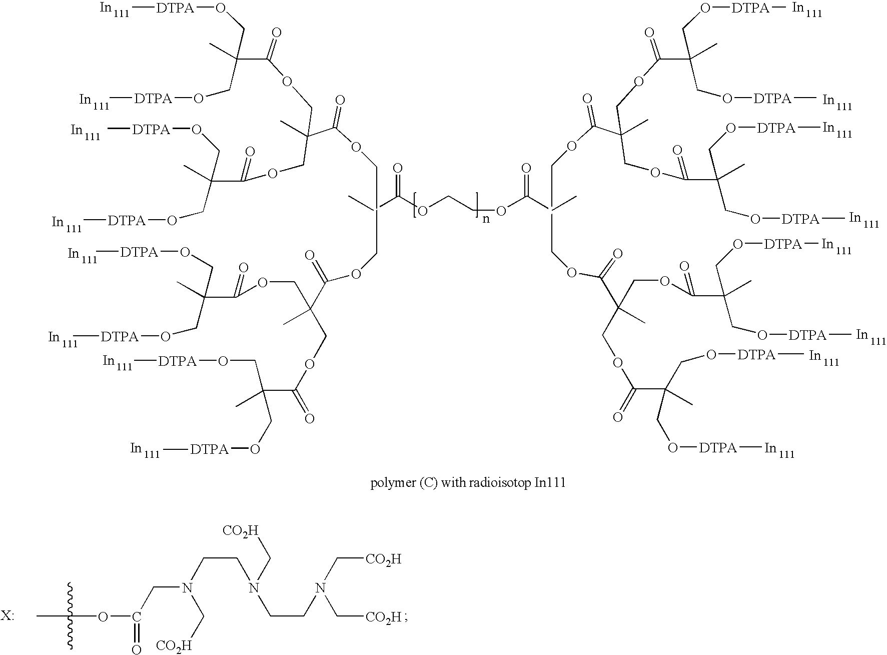

At first, compound (V) (2.88 g, 0.40 mmol) is mixed with dimethylaminopyridine (DMAP, 0.3151 g, 2.57 mmol) in a round-bottomed conical flask followed by dissolving 35 ml dichloromethane (DCM) in nitrogen atmosphere and filling it in the flask. Thereafter, 5.48 g (12.8 mmol) Benzylidene-2,2-bis(oxymethyl)propionic anhydride dissolved in another flask is gradually added in the reacting round-bottomed conical flask recited above. After stirring for 24 hours in the room temperature, 15 ml methanol is added and the reaction is continued for 6 hours to remove un-reacted Benzylidene-2,2-bis(oxymethyl)propionic anhydrides. Subsequently, overdose (700 mL) ether is also added and continuously stirred to form compound (VII), which is white precipitant and gets yield of around 89%. Physical measurement of the compound (VII) is listed below:

1H NMR (400 MHz, CDCl3): δ 0.89 (s, 24), 1.16 (s, 6), 1.17 (s, 12), 3.57 (t, 6), 3.67 (bs), 3.77 (t, 3), 4.15 (q, 6), 4.28 (t, 3), 4.33 (m, 16), 4.55 (d, 16), 5.37 (s, 8), 7.30 (m, 24), 7.35 (m, 16).

Thereafter, 4 g compound (VII) is dissolved in 45 mL DCM/MeOH(1:1) followed by adding 0.4 g Pd/C and stirring for 24 hours in the saturated hydrogen atmosphere. After the reaction is complete, Pd/C is filtered from DCM. A white precipitant product is rapidly obtained via washing by overdose (600 mL) ether. Thus, a compound (VIII) of 1.8 g is formed by cooling and drying. Physical measurement of the compound (VIII) is listed as below:

IR(cm−1): 3401,2887, 1727, 1108.

1H NMR (400 MHz, CDCl3): δ 1.07 (s, 24), 1.27 (s, 6), 1.34 (s, 12), 3.47 (t), 3.64 (bs), 3.76 (m), 4.26 (m), 4.32 (dd, 10).

Thereafter, 1.097 g (0.1938 mmol) compound (VIII) and 1.814 g (3.6 mmol) diethylenetriaminepentaacetic acid mono-N-hydroxysuccinimide ester (DTPA-HSIE) are disposed in a round-bottomed conical flask of 50 mL dried by vacuum for 4 hours. Then, 10 mL anhydrous dimethyl sulfoxide (DMSO) and 515 mL Triethylamine are injected followed by continuous stirring for 48 hours in the saturated hydrogen atmosphere. Free DTPA-HSIE is removed by dialysis, spec. of which is MW1000. 1000 mL deionized water is provided each time as dialysis solvent to keep monitoring DTPA-HSIE until it is absent in the dialysis solvent. Ultimately, a compound (IX) is formed as a white solid product after cooling and drying and physical measurement thereof is listed below:

IR(cm−1): 3460, 2990, 2650, 1720, 1645, 1235.

1H NMR (400 MHz, CDCl3): δ 1.03 (s), 1.25 (s), 1.29 (s), 2.7 (m), 3.16 (t), 3.46 (t), 3.79 (bs), 3.80 (m), 3.97 (bs), 4.21 (m).

Finally, the compound (IX) is dissolved by water to form a solution with concentration of 0.46×10⁻¹⁰/L. Thereafter, In-111-acac 66 μCi (dissolved in ethanol) is added in the solution followed by evacuating ethanol with nitrogen. The water bath is processed for 20 mins in the temperature of 70° C. Subsequently, reverse phase Cl8-plus Sep-Pak is executed for separation and purification, and a polymer (C) containing radioisotope In₁₁₁ is then formed.

### EXAMPLE 4

preparation of polymer (D)

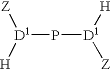

containing radioisotope In₁₁₁:

P: polyethylene glycol segment (molecular weight˜4000);

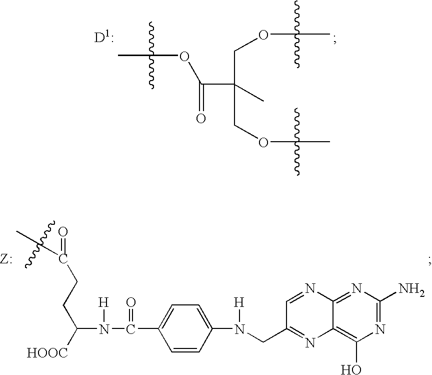

H: hydrogen atom;

Wherein the preparation procedure of the polymer (D) containing the radioisotope is shown as below:

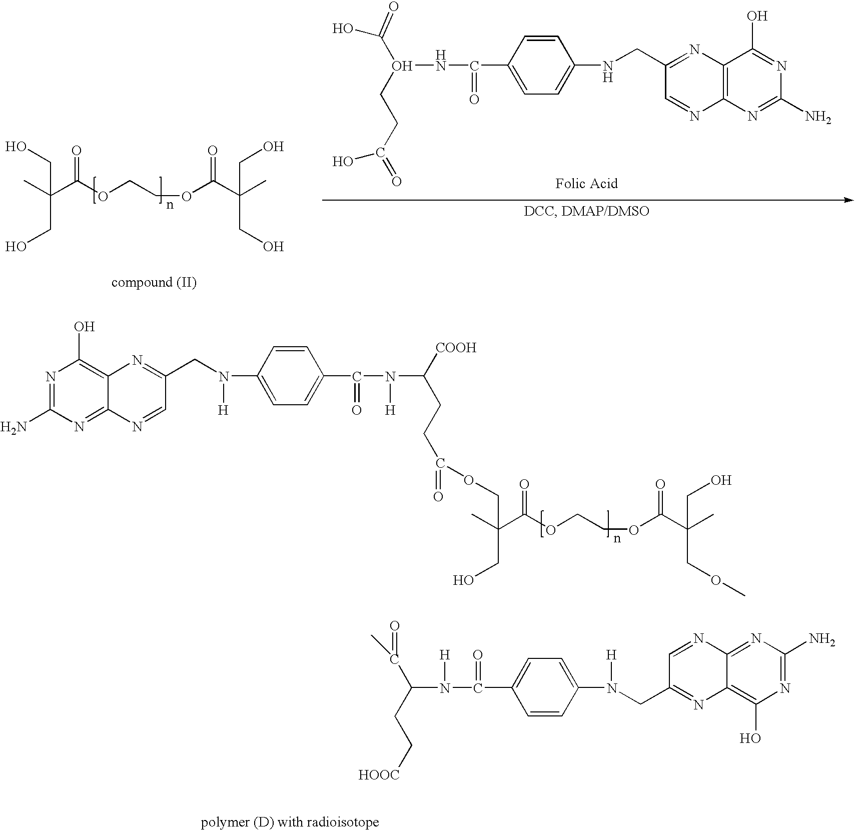

At first, compound (II) (0.3 g, 0.064 mmol), dimethylaminopyridine (DMAP, 0.0252 g, 0.27 mmol), folate (0.11 g, 0.256 mmol), and N,N-Dicyclohexylcarbcdimide (DCC, 0.053 g, 0.26 mmol) are disposed in a round-bottomed conical flask of 50 mL dried by vacuum for 3 hours. Then, 20 mL anhydrous dimethyl sulfoxide (DMSO) is injected followed by continuous stirring for 48 hours in a nitrogen saturated atmosphere. Free DTPA-HSIE is removed by dialysis, spec. of which is MW1000. 1000 mL deionized water is provided each time as dialysis solvent to continue monitoring DTPA-HSIE until it is absent from the dialysis solvent. Ultimately, a compound (D) is formed as a yellow solid product after cooling and drying and physical measurement thereof is listed below:

1H NMR (400 MHz, DMSO-d6): δ 1.03 (s, 6H, —CH3), 1.68-1.80 (m, 8H, —CO—CH2-CH2-CH), 3.49 (m, PEG-O—CH2-), 4.08 (s, 4H, —CH2-NH), 4.62 (m, 2H, —CH—COOH), 4.74 (bs, 2H, —OH), 6.58 (d, 4H, J =8.8 Hz, ArH), 7.40 (d, 4H, J =8.8 Hz, ArH), 8.07 & 8.09 (2s, 4H, NH), 8.65 (s, 2h, pyri-ArH).

Accordingly, the disclosure introduces a concept of dendritic polymer into a design of carriers capable of carrying a plurality of radioisotopes and analyte-specific moieties, so as to increase the signal intensity and specific targeting ability of imaging agent per molecule. In addition, the dendritic molecule recited in the embodiments can avoid developing agent with small molecules easily breaking through the skin cell in the blood and the drawback of easy metabolism by the human body, thus increasing the time present in the blood circulation.

One of the technical characteristics of the disclosure is to design a carrier capable of carrying multiple radioisotopes and analyte-specific moieties by taking poly vinyl glycerol as a core base. Poly vinyl glycerol, a bio-compatible polymer identified by Food and Drug Administration (FDA), is often adopted to modify biomedical polymer materials, which can be expelled from human body via general circulation, further decreases bio toxicity of developing agents and increases bio-compatibility.

In addition, another technical characteristic of the disclosure is a multiple dendritic polymer carrier carrying a plurality of radioisotopes and analyte-specific moieties. Compared with conventional developing agents with big molecular carriers, such as (blood serum protein or ploy phosphoric acid), the dendritic polymers process a unique magnifying ability with geometric series, so as to greatly increase signal strength of per unit nuclear molecular imaging contrast agents.

Compared with the nuclear molecular imaging contrast agents with small molecules (6-7), conventional nuclear molecular imaging contrast agents belong to the categories of chemical compounds with small molecular weight, which has a strong ability to break through human vascular endothelial cells (HEVC). Thus, the nuclear molecular imaging contrast agents easily flow away, are broadly distributed in the circulation process and can not reach the target location. In addition, after these contrast agents with small molecular weight enter a living organism, they are easily metabolized by the human body and can not reach the developing object. Moreover, even if the developing agents with small molecular weight reach the developing location, the developing agents can reach the effect of the developing only with certain numbers of accumulated developing agents.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A nucleus molecular imaging contrast agent, comprising polymers with structures as formula (I), formula, (II): 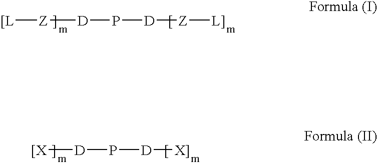
Wherein P is 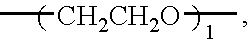
and 1 is not less than 1;
D is independent and comprises a C3-30 dendritic moiety with n oxygen residual groups, wherein n is not less than 3, and D respectively bonds with P and Z by the oxygen residual groups;
Z is independent and comprises a C3-20 moiety having a plurality of functional groups, wherein the functional groups are selected from a group consisting of carbonyl, carboxyl, amine, ester, amide, or chelate group, and Z respectively bonds with D and L by the individual functional group;
L is independent and comprises a radioisotope or analyte-specific moiety;
m is independent and not less than two, and m equals n−1; and
at least one X is an analyte-specific moiety, the other X are hydrogen atoms.

2. The nucleus molecular imaging contrast agent as claimed in claim 1, wherein D comprises 2,2-dihydroxymethyl propanoic acid and residual groups of the derivative thereof.

3. The nucleus molecular imaging contrast agent as claimed in claim 1,
wherein D is 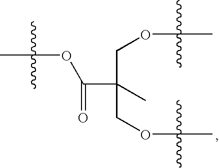
wherein n is 3.

4. The nucleus molecular imaging contrast agent as claimed in claim 3,
wherein D is 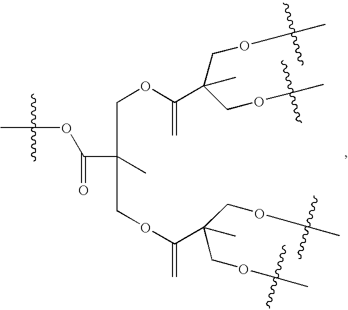
wherein n is 5.

5. The nucleus molecular imaging contrast agent as claimed in claim 3, wherein
D is 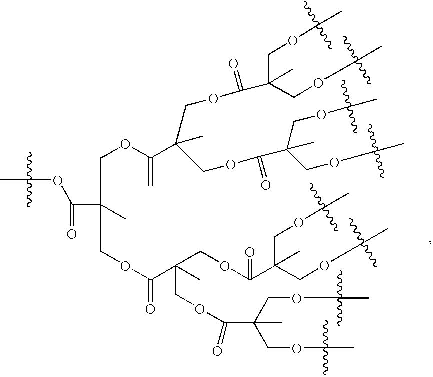
wherein n is 9.

6. The nucleus molecular imaging contrast agent as claimed in claim 1, wherein L is a radioisotope.

7. The nucleus molecular imaging contrast agent as claimed in claim 6, wherein L is In-111.

8. The nucleus molecular imaging contrast agent as claimed in claim 6, wherein L is Gd-153.

9. The nucleus molecular imaging contrast agent as claimed in claim 1, wherein the analyte-specific moiety is a folic acid group.

10. The nucleus molecular imaging contrast agent as claimed in claim 1, wherein the analyte-specific moiety is a glucose group.

11. The nucleus molecular imaging contrast agent as claimed in claim 1, wherein the analyte-specific moiety is an amino acid group.

12. The nucleus molecular imaging contrast agent as claimed in claim 1, wherein the analyte-specific moiety is a deoxyibouncleic acid group.

13. The nucleus molecular imaging contrast agent as claimed in claim 1, wherein Z comprises a metal-chelated group.

14. The nucleus molecular imaging contrast agent as claimed in claim 1, wherein Z comprises a residual group of ethylenedinitrilo tetraacetic acid (EDTA) or a residual group of ethylenediimino dibyric acid (EDBA).

15. The nucleus molecular imaging contrast agent as claimed in claim 9, wherein Z is a residual group of diethylene triaminepentaacetic acid (DTPA).
